(19) United States
(12) Patent Application Publication
(10) Pub. No.: US 2015/0272475 A1
(43) Pub. Date: Oct. 1, 2015

(54) DEVICE FOR THE MEASUREMENT AND ANALYSIS OF THE MULTIPLE BREATH NITROGEN WASHOUT PROCESS

(71) Applicant: ndd Medizintechnik AG, Zurich (CH)

(72) Inventor: Christian BUESS, Horgen (CH)

(21) Appl. No.: 14/675,569

(22) Filed: Mar. 31, 2015

(30) Foreign Application Priority Data
Apr. 1, 2014 (DE) 102014004765.2

(51) Int. Cl. A61B 5/08; A61B 5/083; A61M 16/08; A61B 5/097; A61B 5/00

(52) U.S. Cl. CPC A61B 5/0813; A61B 5/097; A61B 5/486; A61B 5/742; A61M 16/0833; A61M 16/085; A61B 5/0836; A61B 5/7225; A61B 2562/0204; A61B 2560/0475; A61M 2202/0208; A61M 2202/0266; A61M 2202/0225; A61B 2560/0223

(57) ABSTRACT

The present disclosure relates to a device for the measurement and analysis of the multiple breath washout process, where an ultrasound, flow and molar mass sensor determines the instantaneous flow and the instantaneous molar mass of the gas inspired and expired by the patient in the main flow.

### CROSS REFERENCE TO RELATED APPLICATION

This application claims priority to German Patent Application No. 10 2014 004 765.2, entitled “Device for the Measurement and Analysis of the Multiple Breath Nitrogen Washout Process”, filed Apr. 1, 2014, which is hereby incorporated by reference in its entirety for all purposes.

### TECHNICAL FIELD

The present disclosure relates to a device for the measurement and analysis of the multiple breath nitrogen washout process.

### BACKGROUND AND SUMMARY

The measurement of the washout procedure of tracer gases from the lung has been carried out since around 1950 and has been used since then to determine the lung volume, subdivisions of the lung volume and parameters of the ventilation inhomogeneity [see: A. A. Hutchison, A. C. Sum, T. A. Demis, A. Erben, L. I. Landau. Moment analysis of multiple breath nitrogen washout in children Am Rev Respir Dis 1982; 125: 28-32]. The multiple breath washout process can be carried out using different tracer gases: Helium, SF6 (sulfur hexafluoride) or N2 (nitrogen) are the most frequently used tracer gases. The use of N2 is the simplest method since the patient only has to be switched from breathing air to breathing 100% oxygen. When breathing oxygen, the N2 remaining in the lung is washed out of the lung breath for breath. Various methods have been developed for measuring the concentrations of the tracer gases N2, SF6 or helium, each having individual advantages and disadvantages.

The analysis using the nitrogen multiple breath washout process (MBW) requires a precise measurement of the flow speed of the gases flowing in and out of the lung combined with an equally precise measurement of the N2 concentrations inspired and expired by the patient. Both the flow speed and the N2 signal have to be measured using fast-response sensors since the flow signals and concentration signals can be subject to fast changes even with basal respiration. In addition the flow signal and the N2 concentration signal have to be recorded synchronously (i.e. without any time delay between the signals) since the product of the N2 concentration and the flow is integrated to determine the inspired and expired N2 volumes.

However, the inventors herein have recognized that most washout systems utilize a combination a main flow measurement of the flow of gases (F) and secondary flow measurement of gas concentration for the multiple breath washout analysis. However, a position of the secondary flow measurement may be positioned a distance away from the patient and the main flow measurement. This results in a time delay between the main and secondary flow measurements, thereby resulting in non-synchronous recording of the flow signal and N2 concentration signal. This results in reduced accuracy of the resulting lung measurements of the patient which may, in turn, result in improper diagnosis.

It is the aim of the present disclosure to provide a device for the measurement and analysis of multiple breath nitrogen washout processes that at least partially addresses the above-described issues. In one example, a system for a multiple breath washout process includes: a first molar mass sensor including an ultrasound transducer disposed in a main flow of gases inspired and expired by a user; a gas sensor disposed in a secondary flow of the gases; a second molar mass sensor disposed in the secondary flow; and a controller with computer readable instructions for: receiving a first molar mass and a gas flow rate from the first molar mass sensor, a concentration of a first gas from the gas sensor, and a second molar mass from the second molar mass sensor; and estimating a concentration of a second tracer gas based on the received first molar mass, gas flow rate, concentration of the first gas, and the second molar mass.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

FIG. 1 shows example graphs for a flow of gas and nitrogen concentration over time during a nitrogen washout process.

FIG. 2 shows a first embodiment of a device for a nitrogen washout process.

FIG. 3 shows a second embodiment of a device for a nitrogen washout process.

FIG. 4 shows a flow chart of a method for performing a multiple breath washout procedure.

### DETAILED DESCRIPTION

In the following descriptions of the figures, the washout process based on nitrogen (N2) (as the tracer gas) will be described. The method can, however, also be used in an analog manner for the analysis of the multiple breath washout using the tracer gases SF6 or helium.

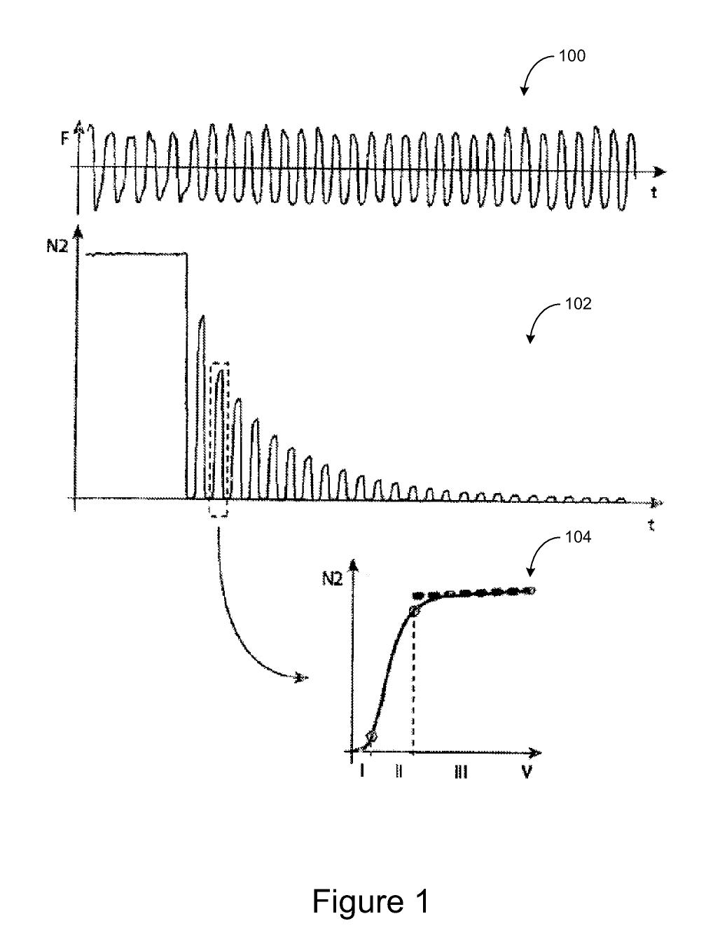

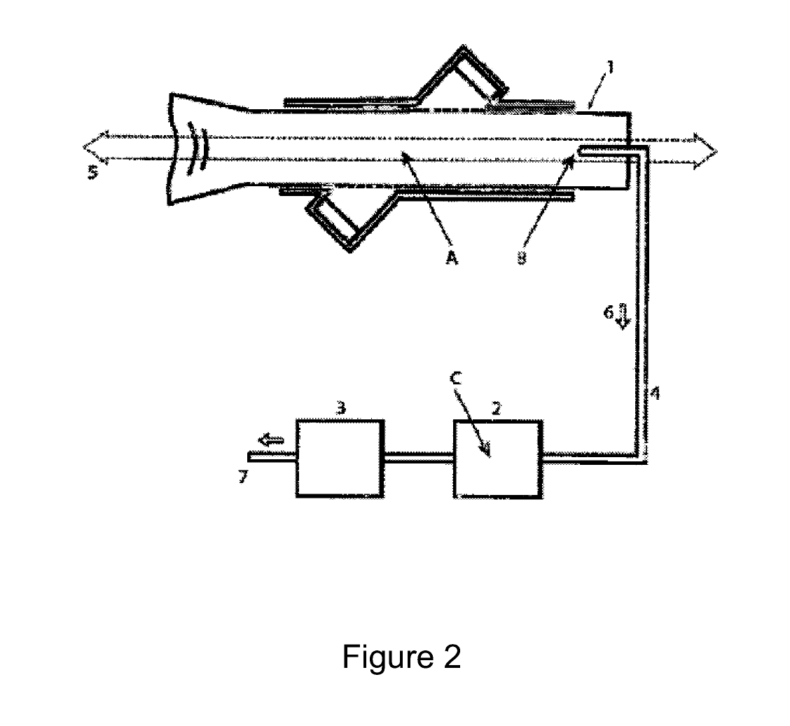

FIG. 1 shows example plots of data resulting from a nitrogen multiple breath washout process (MBW) where a patient is breathing 100% oxygen with a test device (e.g., such as the test device shown in FIG. 2). More specifically, FIG. 1 shows a first plot 100 of the flow (F) of gases (e.g., flow rate or flow speed of gasses) into and out of a patient's lungs (e.g., the inspired and expired flow) over time (t) and a second plot 102 of the nitrogen (N2) concentration of the gases inspired and expired by the patient over time (t). The patient breathes, into a mouthpiece, normally during the entire MBW test, but with an attached nose clamp so that all the air is inspired and expired through the mouth. The N2 concentration is almost constant prior to the actual washout process, which starts at time t1, and is at the value of the environmental N2 concentration (78.08%); during the washout process, the N2 concentration during inspiration is (almost) zero and during expiration the mean N2 concentration decreases with every breath since the nitrogen is further washed out of the lung with each breath. FIG. 1 likewise includes an enlarged plot 104 of the N2 concentration over the expired volume (V). This representation illustrates phases I to III of an expiration (I=dead space; II=mixed air; III=alveolar air). Further parameters for quantifying the ventilation inhomogeneity can be determined by the determination of the increase in phase III of all breaths of the washout process [Consensus statement for inert gas washout measurement using multiple and single breath tests. Eur Respir J 2013; 41: 507-522].

The flow speed (F) and the gas concentration signals (e.g., N2 concentration) are typically detected using a measurement rate of 100 Hz. Flow sensors typically have a response time <10 ms; the sensors for measuring gas concentrations are usually slower and have response times in the range from 60 to 100 ms (see [Consensus statement for inert gas washout measurement using multiple and single breath tests. Eur Respir J 2013; 41: 507-522]). Different types of flow sensors for gases are available such as sensors which detect the pressure difference via a resistance in the flow path or sensors which measure the cooling of a hot-wire, sensors which measure the rotation of a turbine or sensors which measure the ultrasound transit time. The gas concentrations are subject to great fluctuations during the washout process. However, this may not have any effect on the flow measurement or this influence has to be sufficiently corrected. However, it is not only the technology of the flow sensors which differs. The methods of how the current systems analyze the washout by measurement of the tracer gas for determining the N2 concentration also differ. The methods described below may be used for measuring the N2 concentration.

Mass spectrometry: Mass spectrometers can be used for measuring the gas concentrations with high precision and with fast response times. In the multiple breath nitrogen washout process, the values of the concentration of N2, O2 and CO2 are typically detected simultaneously. Mass spectrometers are used most frequently when SF6 is used as the tracer gas for the measurement of the multiple breath washout [P. M. Gustafsson, P. Aurora, A. Lindblad. Evaluation of ventilation maldistribution as an early indicator of lung disease in children with cystic fibrosis. Eur Respir J 2003; 22: 972-979]. Mass spectrometers are very expensive and have to be serviced regularly.

Indirect process: The N2 concentration can be determined indirectly by the use of two separate gas sensors for measuring the concentrations of O2 and CO2. O2 is usually measured using an electrochemical sensor or with the aid of laser light by IR absorption; IR absorption sensors are typically used for the CO2 measurement. The N2 concentration is determined indirectly with reference to the concentration of O2 and CO2 by applying Dalton's Law according to which the sum of all gases of a mixture produces 100%. To ensure a precise determination of the N2 concentration, the O2 signals and CO2 signals have to be measured simultaneously, that is without any time delay between the signals. The sensors furthermore have to have response times which are as similar as possible [F. Singer, B. Houltz, P. Latzin, P. Robinson, P. Gustafsson. A Realistic Validation Study of a New Nitrogen Multiple-Breath Washout System PloS ONE 7(4): e36083, 2012].

If the synchronous signals of the N2 concentration and of the gas flow are present, the expiratory total nitrogen volume is determined by mathematical integration of the inspiratory and expiratory N2 volumes multiplied by the flow. The inspired and expired N2 volume of each breath is therefore determined and these volumes are added to determine the total expired N2 volume until a threshold value for N2 concentration is reached (typically <2.5% of the initial concentration). Finally, the total expired N2 volume is divided by the initial N2 concentration to calculate the functional residual capacity (FRC) and the final value (threshold value) of the N2 concentration is in turn subtracted from it [Consensus statement for inert gas washout measurement using multiple and single breath tests. Eur Respir J 2013; 41: 507-522]. Parameters of the ventilation inhomogeneity (such as the LCI and moments) are determined by a more detailed analysis of the nitrogen concentration over time or over the volume.

The present disclosure uses the measurement of flow and molar mass by ultrasound [cf. EP 0 597 060 B1, EP 0 653 919 B1] in combination with a fast-response CO2 sensor, typically on the basis of an IR absorption process, for determining the functional residual capacity (FRC), the lung clearance index (LCI), the moment ratios, the phase III analysis (including Scond and Sacin) as well as further derived parameters of a multiple breath nitrogen washout test.

The following gas components have to be taken into account before the actual washout phase when the patient is breathing room air and during the washout phase when the patient is breathing 100% oxygen.

N2 (nitrogen); the concentration is approximately 78.08% in (dry) room air.

O2 (oxygen); the concentration is approximately 20.95% in (dry) room air %.

CO2 (carbon dioxide); the concentration is approximately 0.04% in (dry) room air; during expiration it increases to approximately 4 to 5%.

H2O (water vapor); the concentration in air lies between 0 and approximately 5% in dependence on the humidity and on the temperature.

Ar (argon); the concentration is approximately 0.93% in (dry) room air.

The concentration is below 0.002% for all other gases (the so-called trace gases). These gases remain out of consideration in the following discussion.

The concentration of all gases fluctuates in the course of inspiration and expiration during the multiple breath washout process. The three following equations can be set up independently of these changes in the gas concentration which are mainly due to the inhalation of oxygen and the exhalation of the created carbon dioxide:

f_{N2}+f_{O2}+f_{CO2}+f_{H2O}+f_{Ar}=1 (1)

f_{N2}M_{N2}+f_{O2}M_{O2}+f_{CO2}M_{CO2}+f_{H2O}M_{H2O}+f_{Ar}M_{Ar}=M (2)

$${f}_{Ar} = {f}_{N 2} \cdot \frac{0.94}{78.2} \qquad ( 3 )$$

Here f x is the proportion of the gas x and Mx is the molar mass of the gas x in g/mol.

Equation (1) shows that the sum of all gas proportions (e.g., gas fractions) observed results in a total fraction of 1 (or 100% if the gas proportions are in percentage form) (Dalton's Law). As stated above, the trace gases are left out of consideration here.

Equation (2) describes the measurement of the molar mass. The molar mass measured by the ultrasound sensor is calculated by summing the proportions of each gas and multiplying them by the respective molar mass.

It is assumed in equation (3) that the proportion of argon is in a fixed ratio with the nitrogen proportion, that is that the argon washout is directly proportional to the nitrogen washout. Since neither of these two gases is involved in the gas exchange during respiration of a person, this is a valid assumption.

The following list shows which variables of equations (1) to (3) are measured and which are unknown:
Measured variables: fCO2, fH2O, M
Unknown variables: fN2, fO2, fAr

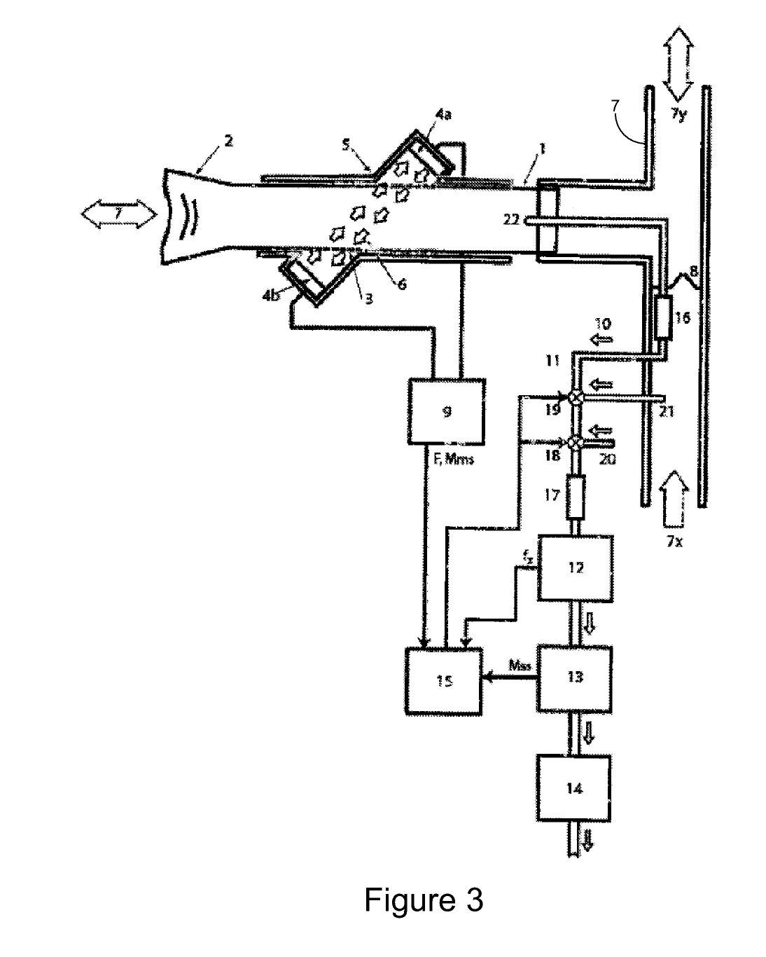

The respective sensors measure the CO2 concentrations (fCO2) and the molar mass (M). The water vapor concentration (fH2O) in the air is determined by measuring the humidity, the pressure and the temperature of the room air. The concentration of the water vapor in room air can be determined from these values. The mechanical design of the present disclosure (see FIG. 3, as described further below) ensures a defined water vapor concentration in the region of the sensor during the total washout process.

Since three unknown variables and three equations are present, the system can be resolved and the concentrations for fN2, fO2 and fAr can be determined. The proportions of fN2 and fO2 can therefore be determined from the measured values for fCO2, fH2O and M.

The listed equations apply to all phases of the nitrogen washout from the lung of a patient; they apply equally to in-vitro systems, that is to systems for validating medical devices for the analysis of the nitrogen washout; these systems can make use of an apparatus such as a syringe and/or a container to simulate the patient's lung (F. Singer, B. Houltz, P. Latzin, P. Robinson, P. Gustafsson. A Realistic Validation Study of a New Nitrogen Multiple-Breath Washout System PloS ONE 7(4): e36083, 2012].

To achieve a better precision, the values for the molar mass x in equation (2) can be replaced by corrected molar mass values M*x, where M*x=kx*Mx and where the factor kx is a dimensionless constant for the adiabatic index correction (that is the thermal capacity ratio). This correction is necessary because the measurement of the molar mass (the total molar mass M in equation (2)) is based on a fixed thermal capacity ratio (see (2)).

To achieve a sufficient precision in the evaluation of the multiple breath washout process, the values for fN2 and fO2 have to be determined with a relatively high precision (the precision of the N2 concentration measurement should be <0.2% [Consensus statement for inert gas washout measurement using multiple and single breath tests. Eur Respir J 2013; 41: 507-522]). In this connection, a cross-sensitivity of the CO2 sensor with oxygen should be taken into account when CO2 is measured with the aid of infrared absorption [R. Lauber, B. Seeberger, A. M. Zbinden. Carbon dioxide analysers: accuracy, alarm limits and effects of interfering gases. Can J Anaesth 1995, 42:7, 643-656]. This additional equation which carries out a correction of the measured CO2 value with the calculated oxygen concentration can be easily added to equations (1) and (3).

In accordance with the present disclosure, the above-described device uses a sensor technology for the measuring of N2 washouts in a new way. The sensors used in the embodiment are inexpensive and have a high long-term stability which allows the construction of a cost-effective device for multiple breath washout analyses which can be utilized for a plurality of patients (newborns to adults) and could also be adapted to a use with patients on ventilators.

Further features, details and advantages of the present disclosure will be described in more detail with references to the embodiment in the drawing of FIG. 3.

Most systems utilize a combination a main flow measurement and secondary flow measurement for the multiple breath washout analysis. FIG. 2 explains this concept: A flow sensor with a flow tube (1) measures the air which flows into the lung of the patient and back out again. The flowing gas is measured at the center of the flow sensor (at the point A) in the main flow (5). Since the gas sensor is too large or because of restrictions in the measurement setup, gas concentrations cannot be measured directly in the main flow (5) in most cases. A small portion of the gas from the main flow is therefore sucked in via a sample tip arranged at the point B. The secondary flow sample (6) is supplied via a hose with the aid of a pump (3) to the gas sensor (2) and is subsequently led off via the outlet (7). The sample hose (4) is usually very long and has a small diameter (e.g. a length of 1 m with a diameter of 1 mm) so that the gas sensor can be positioned at a distance from the patient. Due to the time delay by the transport from point B, at which the secondary flow sample enters into the hose, to the point C, where the gas concentration is measured, the gas flow signals of points A and C are no longer synchronous. The synchronicity is, however, a requirement for an exact multiple breath washout analysis [Consensus statement for inert gas washout measurement using multiple and single breath tests. Eur Respir J 2013; 41: 507-522]. There is furthermore typically also a spacing between the point A of the flow measurement and the point at which the secondary flow sample is taken. The time delay between the flow signal and the gas concentration signal additionally depends on the speed and direction of the main flow and not only on the speed of the secondary flow sample due to this distance. The two time delays are compensated by the embodiment of the present disclosure, described below with reference to FIG. 3.

FIG. 3 shows the block diagram of an embodiment of a system for the multiple breath nitrogen washout analysis that may reduce inaccuracies in the determined nitrogen concentration by reducing measurement time delays and providing a means of calibrating the washout device sensors. The patient breaths into an ultrasound flow and molar mass sensor (5). The inspired and expired air (7) flows through the respiration tube (1) having a mouthpiece (2), which can be replaced and disposed of, to avoid any cross-contamination between patients. The flow speed of the air and the molar mass in the respiration tube are determined with the aid of the ultrasound transit time measurement: Two ultrasound transducers (4a, 4b) transmit ultrasonic pulses (6) in the respiration tube with and against the respiratory flow. The ultrasonic pulses pass through the fine tissue (3) in the respiration tube. An electronic unit (9) triggers the transmission of the pulses, measures the transit times of the ultrasonic pulses upstream and downstream (4a) and (4) and calculates the flow speed (F) and the molar mass (Mms) in the main flow using these transit times [EP 0 597 060 B1, EP 0 653 919 B1]. The results for the flow and for the molar mass in the main flow are forwarded from the electronic unit (9) to the main processing unit (15). In the current embodiment, the measuring and transmission frequency of these signals to the processing unit (15) is at 200 Hz.

The end of the respiration tube (1) is inserted into a T tube (7). In the phase before the actual washout, the patient inhales and exhales air via a connection (′7y) of the T tube (7) which is open to the room air. A one-way valve (8) ensures that no gas is inhaled from the other part of the T tube. If a switch is made to oxygen inspiration, i.e. if the actual nitrogen washout takes place using the multiple breath process and oxygen (e.g., washout gas) is supplied to the main flow (e.g., the main flow of gases inspired and expired by the patient in the respiration tube (1), the patient inspires oxygen which is supplied via the connection (7x) of the T tube (7) at a constant or variable speed and expires again via the connection (7y). The flow of the oxygen supplied at the connection (7x) has to be larger than the maximum inspiration flow of the patient. Oxygen can be provided via the wall port in the hospital and otherwise from an oxygen bottle. The patient therefore only inspires oxygen from the transverse flow (e.g., flow flowing perpendicular to the respiration flow of inspired/expired air in the respiration tube) during the washout phase; the patient does not inspire any room air during the washout phase of the test. A sample is taken from the secondary flow (10) for the gas analysis. A pump (14) conveys the gas sample (10) through a sample hose (11) to the CO2 sensor (12) and to the molecular mass sensor (e.g., second molar mass sensor) (13).

For the gas analysis, the gas sample has to flow out of the secondary flow through the sample hose to arrive at the gas analyzers for the measurement of the molar mass via the molar mass sensor (13) and of the CO2 via the CO2 sensor (12). The molar mass sensor (13) and the CO2 sensor (12) are positioned proximate to one another and both sensors are positioned fluidly away from the molar mass sensor (5). Said another way, the molar mass sensor (13) and CO2 sensor (12) are positioned a distance away from the molar mass sensor (5) such that fluid has to travel a distance from the molar mass sensor (5) before reaching the molar mass sensor (13) and CO2 sensor (12). As a result, there is a delay between the time when the molar mass sensor (5) measures a gas and when the molar mass sensor (13) and CO2 sensor (12) measures the same gas. The delay caused by the gas transport depends on various variables (e.g. on the environmental pressure, on the pump speed, etc.) and it can also differ from test to test due to slight changes in the test setup. If, for example, the sample hose (11) used is a replaceable part, the diameter and length can vary slightly from test to test. The inspired and expired N2 volumes have to be determined for the multiple breath washout analysis. This is done by multiplying the nitrogen concentration by the flow speed of the main flow and a subsequent integration over time. The correct function of this process is only ensured when the flow signal and the N2 concentration signal are synchronous; time delays between the signal of the flow speed of the main flow and the gas signals of the secondary flow result in a determination of the inspired and expired gas volumes suffering from errors [Consensus statement for inert gas washout measurement using multiple and single breath tests. Eur Respir J 2013; 41: 507-522]. The molar mass signal measured by molar mass sensor (13) and the CO2 signal measured by CO2 sensor (12) therefore have to be synchronized with the flow speed signal which is measured at the center of the respiration flow in the respiration tube (1), between the two ultrasound transducers (4a) and (4b). The synchronization can be achieved by cross-correlation of the first molar mass signal from the main flow (Mms) via the first molar mass sensor (5) and the second molar mass signal via the second molar mass sensor (13) from the secondary flow (Mss). This process is described in the patent [EP 1 764 036 B1]; an application option for the CO diffusion measurement SB (single breath inspiration method) is described in the patent [EP 1 764 035].

The processing unit (15) controls two valves (18) and (19). These two valves serve to switch over the secondary flow current between the sample connections (22), (21), and (20). The sample connection (22) is the primary sample connection which is utilized during the test. The sample connection (21) is only utilized during the calibration phase of the test. Samples of the gas which is supplied to the patient during the washout phase (100% oxygen) are taken via this connection. This is a first calibration point for the molar mass sensor (13). The sample connection (20) is also only utilized during the calibration phase of the test. Samples of the room air are taken via this connection. This gas serves as a second calibration point for the molar mass sensor (13) and as a zero reference for the CO2 sensor (12).

Further elements in the secondary flow serve the treatment of the gas before it enters into the gas sensors. Hose material which is permeable for water vapor (16), e.g. with a Nafion® membrane, is used to reduce the moisture (water vapor) of the secondary flow gas. The hose (16) is typically positioned in the proximity of the sample connection 22 in the region of the oxygen feed as this reduces the risk of water condensation in the secondary flow region of the system as the air expired by the patient is saturated with 100% water vapor. The positioning within the oxygen feed (e.g., within a section of the T tube flowing oxygen and coupled to the connection (7x)) very substantially reduces the water vapor quantity since the oxygen introduced is free of water vapor. Further hose material which is permeable for water vapor (17) can be used so that the secondary flow current has a defined water vapor partial pressure on entry into the gas sensors. This additional hose material permeable for water vapor ensures a uniform water vapor partial pressure of all measured gas samples of the secondary flow from the connections 20, 21 and 22, and indeed independently of the fact that the measured gas of the one source is free of water vapor (gas from connection 21), the measured gas of the second source has the humidity of the room air (connection 20) and the water vapor content of the third source fluctuates (connection 22).

The main processor (15) may be referred to herein as a controller (e.g., electronic controller) of the multiple breath washout system. As such, the controller includes non-transitory memory for storing data and instructions for carrying out a multiple breath washout test (e.g., procedure) using the multiple breath washout system components described above. The controller may communicate with various sensors and actuators of the multiple breath washout system. For example, the controller may receive signals from the molar mass sensor (5) via the electronic unit (9), the CO2 sensor (12), and the second molar mass sensor (13). Additionally, the controller may employ various actuators to adjust system components based on received signals. For example, the controller may adjust, via one or more actuators, a position of valves 18 and 19 and/or a flow of gas (e.g., 02) through the connection (7x) of the T tube (7).

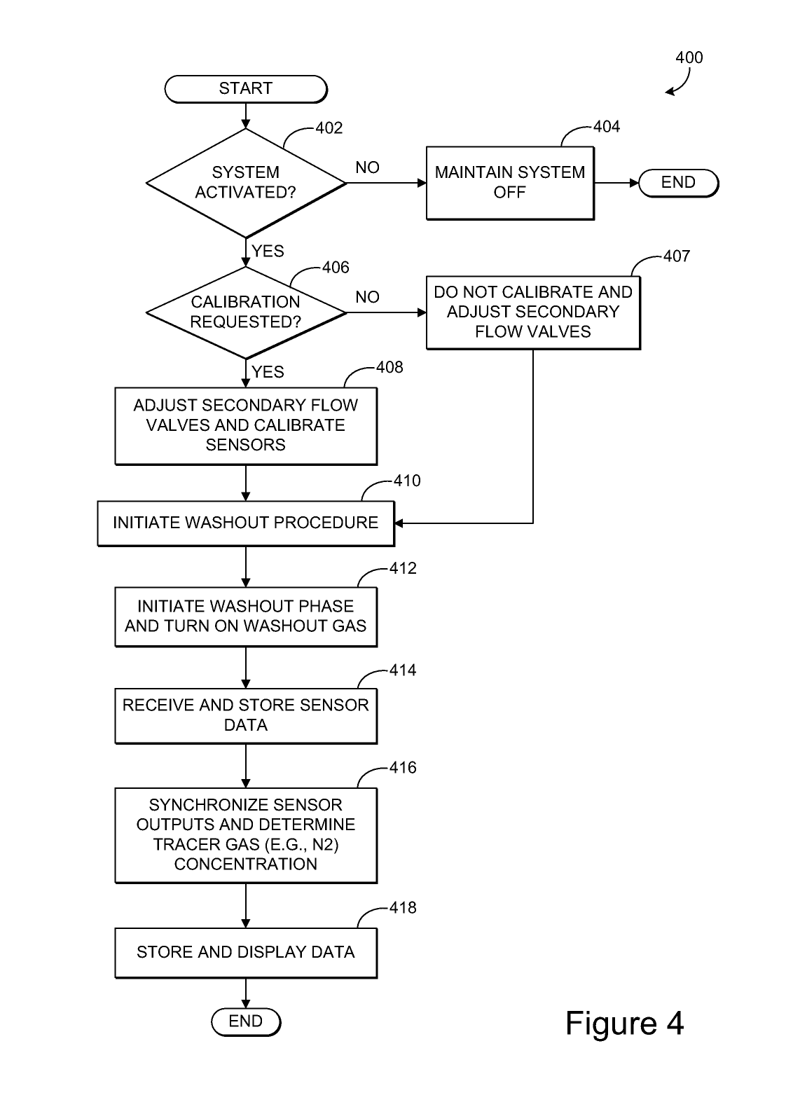

In alternate embodiments, the system shown in FIG. 3 may utilize a different washout gas (other than O2) and/or different gas sensors (other than the CO2 sensor), such as an O2 sensor, to measure the secondary flow gas concentration. As such, the tracer gas may be a gas other than N2. However, the system setup shown in FIG. 3 and the washout method, as shown in FIG. 4, may be substantially the same for these alternate embodiments.

FIG. 4 shows a method 400 for performing the multiple breath washout process using a multiple breath washout system, such as the system shown in FIG. 3. A controller, such as the main processor (15) shown in FIG. 3 may execute method 300 based on instructions stored on the controller memory, in combination with various sensors and actuators of the system, as described above with reference to FIG. 3.

Method 400 starts at 402 by determining if the multiple breath washout system has been activated. In one embodiment, the system may be activated when the controller receives a signal requesting a multiple breath washout test. If the controller does not receive a signal activating the system or washout process, then the method continues to 404 to maintain the system off and not implement the washout procedure. Otherwise, if the controller receives an activation signal at 402, the method continues to 406 to determine if system calibration is requested. As one example, calibration of the washout system may be performed prior to each data acquisition for each patient. As another example, calibration of the washout system may be performed once at system start-up and the calibration values may be used for a series of tests with one or more patients.

If system calibration is not requested, the method continues to 407 to not calibrate the sensors and instead maintain the secondary flow valves (e.g., valves 18 and 19 of FIG. 3) in a position that only allows secondary flow to enter the secondary flow sample hose from the sample connection (e.g., sample port) positioned within the main flow (e.g., within respiration tube 1 shown in FIG. 3). The method then continues to 410, as described further below.

Alternatively, if system calibration is requested, the method continues to 408 to adjust the secondary flow valves (e.g., valves 18 and 19 of FIG. 3) and calibrate the system, as described above with reference to FIG. 3. For example, the method at 408 may include adjusting the valves (e.g., closing valve 18 to ambient air and opening valve 19 to washout gas) to receive a secondary flow of washout gas (e.g., the 100% oxygen used during the washout phase of the test) via a second sample connection (e.g., sample connection 21 shown in FIG. 3) at the second molar mass sensor (e.g., molar mass sensor 13 shown in FIG. 3) disposed in the secondary flow sample hose. The oxygen flow may be sampled by the second molar mass sensor and the received sensor signal may be used by the controller to determine a first calibration point for the second molar mass sensor. The method at 408 may further include adjusting the valves (e.g., closing the valve 19 to washout gas and opening valve 18 to ambient air) to receive a secondary flow of ambient air (e.g., room air) via a third sample connection (e.g., sample connection 20 shown in FIG. 3) at the CO2 sensor (e.g., CO2 sensor 12 shown in FIG. 3) disposed in the secondary flow sample hose. The ambient air flow may be sampled by the CO2 sensor and the controller may calibrate the CO2 sensor based on the received signal from the CO2 sensor. Additionally, the ambient air flow may be sampled by the second molar mass sensor and the received sensor signal may be used by the controller to determine a second calibration point for the second molar mass sensor. The controller then calibrates the second molar mass sensor based on the first and second calibration points. As described above, receiving a secondary flow of ambient air may include receiving only ambient air and not washout gas or inspire/expired main flow air at the sensors in the secondary flow line. Similarly, receiving a secondary flow of washout gas may include receiving only the washout gas and not ambient air or inspired/expired main flow air at the sensors in the secondary flow line.

After calibration is complete, or if calibration is not requested at 406, the method continues to 410 to initiate the washout procedure. Initiating the washout procedure may include maintaining the flow of oxygen through the T tube off, activating the secondary flow pump, beginning to acquire data via the system sensors (e.g., the first ultrasound molar mass sensor measuring gas flow rate and molar mass, the CO2 sensor, and the second molar mass sensor), and storing the acquired data in a memory of the controller. After a duration, or in response to a user input, the method continues to 412 to initiate the washout phase by activating the flow of washout gas (e.g., oxygen) through the T-tube. In alternate embodiments, the method at 412 may include activating the flow of a different washout gas, other than oxygen. At 414, the method includes receiving and storing data measured by the system sensors in the controller memory. At 416, the method includes synchronizing the system sensor outputs and determining the N2 concentration of the main flow of inspired and expired gas flow based on the CO2 concentration and the gas flow speed, as determined from the output of the ultrasound molar mass sensor in the main flow. For example, the method at 416 includes first synchronizing the CO2 signal measured via the CO2 sensor with the flow speed signal measured via the ultrasound molar mass sensor via cross-correlation of the first molar mass signal from the main flow and the second molar mass signal from the secondary flow. This synchronization eliminates time delays due to the different locations of the main flow sensor and the CO2 sensor. The N2 concentration is then determined based on the synchronized CO2 and gas flow speed measurements. At 418, the method includes storing the received and processed data in the memory of the controller and displaying the resulting data to a user (e.g., via a display). For example, the determined N2 concentration over time or over expired volume may be displayed to a user at 418. Further, storing the data may include logging the determined N2 concentration over time and additional lung data determined from the test data in a database. The additional lung data may include functional residual capacity (FRC), lung clearance index (LCI), moment ratios, phase III analysis (including Scond and Sacin), as well as further derived parameter of the multiple breath nitrogen washout test. In this way, the method at 418 may include determining the additional lung data based on the determined N2 concentration.

The innovation of the described analysis system for the multiple breath washout process comprises the calculation of the nitrogen concentration on the basis of the signals of a molar mass sensor and of a conventional infrared CO2 sensor. A further innovation is the automatic calibration of the gas sensors with room air and with the washout gas (oxygen). The system which calculates the nitrogen concentration on the basis of the signals of a molar mass sensor and of a conventional O2 sensor can have a similar structure.

The control methods and routines disclosed herein may be stored as executable instructions in non-transitory memory of the controller and carried out by the controller in combination with the various structural system elements, such as actuators, valves, etc. The specific routines described herein may represent one or more of any number of processing strategies such as event-driven, interrupt-driven, multi-tasking, multi-threading, and the like. As such, various actions, operations, and/or functions illustrated may be performed in the sequence illustrated, in parallel, or in some cases omitted. Likewise, the order of processing is not necessarily required to achieve the features and advantages of the example embodiments described herein, but is provided for ease of illustration and description. One or more of the illustrated actions, operations and/or functions may be repeatedly performed depending on the particular strategy being used. Further, the described actions, operations and/or functions may graphically represent code to be programmed into non-transitory memory of the computer readable storage medium in the engine control system carried out in combination with the described elements of the structural system.

It will be appreciated that the configurations and routines disclosed herein are exemplary in nature, and that these specific embodiments are not to be considered in a limiting sense, because numerous variations are possible.

1. A system for a multiple breath washout process, comprising:
a first molar mass sensor including an ultrasound transducer disposed in a main flow of gases inspired and expired by a user;
a gas sensor disposed in a secondary flow of the gases;
a second molar mass sensor disposed in the secondary flow; and
a controller with computer readable instructions for:
receiving a first molar mass and a gas flow rate from the first molar mass sensor, a concentration of a first gas from the gas sensor, and a second molar mass from the second molar mass sensor; and
estimating a concentration of a second tracer gas based on the received first molar mass, gas flow rate, concentration of the first gas, and the second molar mass.

2. The system of claim 1, wherein the first gas is different than the second tracer gas, wherein the gas sensor is positioned fluidly away from the first molar mass sensor and wherein the second molar mass sensor is positioned proximate to the gas sensor in the secondary flow.

3. The system of claim 2, wherein estimating the concentration of the second tracer gas includes first adjusting the received gas flow rate and concentration of the first gas based on the first molar mass and the second molar mass and estimating the concentration of the second tracer gas based on the adjusted gas flow rate and concentration of the first gas.

4. The system of claim 1, wherein the computer readable instructions further include instructions for displaying the estimated concentration of the second tracer gas to a user.

5. The system of claim 1, wherein the computer readable instructions further include storing the estimated concentration of the second tracer gas in a memory of the controller.

6. The system of claim 1, further comprising:
a respiration tube containing the main flow and including a first end coupled to a mouthpiece and a second end coupled to a T-tube, where a first connection of the T tube is exposed to ambient air and a second connection of the T tube is coupled to a flow of washout gas, and where the first molar mass sensor is disposed in the respiration tube; and
a secondary flow sample hose containing the secondary flow and including a first end disposed within and at the second end of the respiration tube, where the gas sensor and second molar mass sensor are disposed within the secondary flow sample hose.

7. The system of claim 6, further comprising a hose material comprising a water-permeable material and disposed within the secondary flow sample hose downstream of the first end of the secondary flow sample hose and upstream of the gas sensor and second molar mass sensor.

8. The system of claim 6, wherein the washout gas is oxygen, the second tracer gas is nitrogen, the first gas is CO2, and the gas sensor is a CO2 sensor.

9. A method for performing a multiple breath washout procedure, comprising:
via a controller of a washout device:
after initiating a flow of washout gas to a main flow inspired and expired by a user via the washout device, receiving a first molar mass and flow signal from a first molar mass sensor disposed in the main flow, a second molar mass signal from a second molar mass sensor disposed in a secondary flow sampled from a portion of the main flow, and a CO2 concentration from a CO2 sensor disposed in the secondary flow, where the second molar mass sensor is disposed proximate to the CO2 sensor in the secondary flow and the CO2 sensor is positioned away from the first molar mass sensor;
adjusting the received flow signal and CO2 concentration based on the first molar mass signal and the second molar mass signal; and
storing a nitrogen concentration of the main flow in a memory of the controller, the nitrogen concentration estimated based on the adjusted flow signal and CO2 concentration.

10. The method of claim 9, further comprising adjusting one or more valves disposed in the secondary flow of the washout device upstream of the second molar mass sensor and the CO2 sensor to calibrate the second molar mass sensor with washout gas and the CO2 sensor with ambient air.

11. The method of claim 10, wherein adjusting the one or more valves to calibrate the second molar mass sensor and the CO2 sensor includes:
adjusting the one or more valves to provide only washout gas via the secondary flow to the second molar mass sensor and generating a first calibration point for the second molar mass sensor; and
adjusting the one or more valves to provide only ambient air via the secondary flow to the CO2 sensor and second molar mass sensor and generating a zero reference for the CO2 sensor and a second calibration point for the second molar mass sensor.

12. The method of claim 9, wherein adjusting the received flow signal and CO2 concentration includes synchronizing the flow signal and CO2 concentration to account for a transport time delay by cross-correlating the first molar mass signal from the main flow and the second molar mass signal from the secondary flow and further comprising displaying the estimated nitrogen concentration to a user.

13. A device for the measurement and analysis of the multiple breath washout process, where an ultrasound, flow and molar mass sensor determines the instantaneous flow and the instantaneous molar mass of the gas inspired and expired by the patient in the main flow; the instantaneous concentration of CO2 and O2 in the secondary flow, a small gas portion taken from the main flow, inspired and expired by the patient is measured by a sufficiently fast gas sensor; the instantaneous molar mass inspired and expired by the patient is measured by a second ultrasound, flow and molar mass sensor in the secondary flow; and the N2 concentration is determined by solving the two following equations:
a) the sum of the gas concentrations for N2, O2, CO2 and H2O is equal to 100% f_{N2}+f_{O2}+f_{CO2}+F_{H2O}+f_{Ar}=100%: and
b) the sum of all gas concentrations for N2, O2, CO2 and H2O multiplied by their molar mass is equal to the measured molar mass of the gas mixture f_{N2}M_{N2}+f_{O2}M_{O2}+f_{CO2}M_{CO2}+f_{H2O}M_{H2O}+f_{Ar}M_{Ar}=M:
for the N2 gas concentration; and wherein the N2 gas concentration calculated using these equations and the measured flow speed of the main flow for each breath are used for determining the following values: the total inspiratory and expiratory gas volumes, the inspiratory and expiratory N2 gas volume and the parameters derived from the calculated N2 curve and/or from the N2 gas volumes and/or the total inspiratory and expiratory gas volumes.

14. The device in accordance with claim 12, wherein the additional gas argon is used in the two equations of claim 1; and wherein the assumption is used as a starting point that the argon concentration is always in a fixed relationship with the nitrogen concentration.

15. The device in accordance with claim 12, wherein the molar mass values of the individual gases Mx are replaced by a molar mass value M*x=kx*Mx, where kx is a dimensionless constant for the gas x for the adiabatic index correction.

16. The device in accordance with claim 12, wherein an O2 cross-sensitivity toward CO2 is compensated in the CO2 measurement in that the equations are complemented by a suitable correction equation and the equations are solved for N2.

17. The device in accordance with claim 12, wherein a first time delay caused by the transport between the flow measurement in the main flow and the gas measurement in the secondary flow is corrected by determining the delay with reference to a mathematical cross-correlation of the molar mass measurement in the main flow and in the secondary flow and wherein an additional time delay is compensated on the basis of the spacing between the center of the flow measurement and the point of the sample removal in the secondary flow by taking account of a delay time, with the latter having been calculated using a function based on the flow speed of the main flow and on the flow direction.

18. The device in accordance with claim 12, wherein the different response times of the gas sensor and of the molar mass sensor are compensated by mathematical deceleration or acceleration of the speed of the gas sensor or of the molar mass sensor to achieve a response time (almost) the same in both sensors.

19. The device in accordance with claim 12, wherein the offset of the CO2 gas sensor of the secondary flow measurement is automatically calibrated using a gas sample taken from the room air and wherein the offset and gain of the molar mass sensor of the secondary flow measurement is automatically calibrated using gas samples from the room air and the washout gas (oxygen).

20. The device in accordance with claim 12, wherein the parameters FRC (functional residual capacity), LCIX (lung clearance index at x % of the initial tracer concentration), moments and moment ratios, phase III increases and derived parameters Scond and Sacin are analyzed on the basis of the calculated N2 data.
